(19) United States
(12) Patent Application Publication
(10) Pub. No.: US 2026/0355419 A1
(43) Pub. Date: Nov. 12, 2026

(54) SYSTEMS, METHODS, AND APPARATUSES FOR DRUG INJECTORS

(71) Applicant: SOLUtion Medical, Inc., Ambler, PA (US)

(72) Inventors: Julia ANTHONY, Yardley, PA (US); Todd WALLACH, Ambler, PA (US); Mikael AVERY, Collingswood, NJ (US); David PALLING, Philadelphia, PA (US)

(73) Assignee: SOLUtion Medical, Inc., Ambler, PA (US)

(21) Appl. No.: 19/130,525

(22) PCT Filed: Nov. 17, 2023
(86) PCT No.: PCT/US2023/080210

§ 371 Date: May 15, 2025

Related U.S. Application Data
(60) Provisional application No. 63/384,385, filed on Nov. 18, 2022.

(51) Int. Cl. A61M 5/20; A61K 31/573; A61M 5/24; A61M 5/32

(52) U.S. Cl. CPC A61M 5/2033; A61M 5/2066; A61M 5/2448; A61M 5/320233; A61K 31/573; A61M 2005/208

(57) ABSTRACT

The disclosure relates to systems and devices for administration of an active agent into a subject. The devices provided include a housing, a spring release, an outer cap separably engaged with the spring release and rotatable with the spring release, a diluent ejector comprising a stopper, a mixing spring adjacent the diluent ejector providing a bias to the diluent ejector within the housing, a powder barrel comprising a first tearable seal and a second tearable seal, and a crown of thorns positioned proximate the second tearable seal and configured to pierce the second tearable seal responsive to translation of the powder barrel proximally within the housing, and a fluid chamber disposed between the stopper and the first tearable seal. Methods of producing an active pharmaceutical agent are also described.

### CROSS REFERENCE TO RELATED APPLICATIONS

This application claims priority to and the benefit of U.S. Provisional Application Ser. No. 63/384,385, entitled “SYSTEMS, METHODS, AND APPARATUSES FOR DRUG INJECTORS”, filed Nov. 18, 2022, which is hereby incorporated by reference in its entirety.

### FIELD OF THE DISCLOSURE

The disclosure relates to a device comprising a needle for administration of an active agent into a subject, where the active agent is in a solid form in one chamber separated from a diluent or pharmaceutically acceptable carrier in another chamber, the device being capable of exposing the active agent to the diluent or pharmaceutically acceptable carrier for reconstitution, mixing or dissolution prior to administration.

### BACKGROUND

Emergency injections have long been utilized in response to any number of forms of extremely time-sensitive, life-threatening events including overdose, anaphylaxis, angioedema or adrenal insufficiency (AI). The immediate need and essential nature of the active ingredients necessitate both accurate and rapid drug delivery. In fact, while certain naloxone auto-injectors and epinephrine-containing auto-injectors offer a more easily manipulated and administered dosing system, steroid injectors, due to their reliance upon reconstitution, while equally important, continue to suffer from cumbersome delivery systems that provide sub-optimal administration devices in terms of drug preparation and delivery.

For instance, certain prior designs required an individual to inject bacteriostatic water or bacteriostatic saline solution into a sterile vial containing dry hydrocortisone powder, where the diluent in an upper vial is connectively attached to a lower vial containing active dry hydrocortisone ingredient. This requires arduous manipulation of disparate or conjoined vials, alike, for mixing, withdrawal, and administration of the reconstituted mixture all within a life-critical, exceedingly short time period. Even in the case of the relatively simpler Act-o-vial® and similar drug mixing systems, they require an average of twelve or more user steps for injection. Accordingly, needs exist for simplified reconstitution devices.

### BRIEF SUMMARY OF THE INVENTION

The disclosure relates to systems, methods, and apparatuses for transportation, storage, reconstitution, and injection of an active pharmaceutical agent, and the disclosure provides solutions to the issues identified above.

One aspect of the present disclosure provides an injector for delivering a solution of an active pharmaceutical agent and a diluent to a subject. The injector includes a housing. The injector includes a spring release. The injector includes an outer cap separably engaged with the spring release and rotatable with the spring release. The injector includes a diluent ejector comprising a stopper. The injector includes a mixing spring adjacent the diluent ejector providing a bias to the diluent ejector within the housing. The injector includes a powder barrel comprising a first tearable seal and a second tearable seal. The injector includes a crown of thorns positioned proximate the second tearable seal and configured to pierce the second tearable seal responsive to translation of the powder barrel proximally within the housing. The injector includes a fluid chamber disposed between the stopper and the first tearable seal.

Another aspect of the present disclosure provides a method of administering an active pharmaceutical agent to a subject through an injector. The method includes twisting an outer cap of the injector to activate mixing of the active pharmaceutical agent with a diluent. The method of activating mixing includes actuating a spring release to release a mixing spring such that the mixing spring provides an axial force onto a diluent ejector to cause the diluent ejector to translate proximally through the injector. The method of activating mixing includes piercing a first tearable seal with the diluent ejector and providing an axial force on a powder barrel such that the diluent ejector and powder barrel translate proximally through the injector toward a crown of thorns. The method of activating mixing includes piercing, via the crown of thorns, a second tearable seal positioned proximal to the diluent ejector and powder barrel, thereby causing the active pharmaceutical agent and the diluent to begin mixing into an at least partially mixed solution. The method of activating mixing includes passing the at least partially mixed solution, via force from the mixing spring, between the crown of thorns and toward a mixing-chamber seal, wherein force of the at least partially mixed solution passing through the crown of thorns causes the mixing-chamber seal to translate proximally and form a mixing chamber in which to fully mix the active pharmaceutical agent and the diluent into a fully-mixed solution.

Another aspect of the present disclosure provides a method of assembling an injector. The method includes inserting a powder barrel comprising an active pharmaceutical agent into a syringe barrel. The method includes adding a diluent to the syringe barrel. The method includes inserting a diluent ejector into the syringe barrel such that the diluent is positioned between the diluent ejector and the powder barrel, thereby creating an aseptic sealed subassembly for the diluent and the active pharmaceutical agent.

These and other aspects of the present invention are described in the Detailed Description below and the accompanying figures. Other aspects and features of examples of the present invention will become apparent to those of ordinary skill in the art upon reviewing the following description of specific, exemplary examples of the present invention in concert with the figures. While features of the present invention can be discussed relative to certain examples and figures, all examples of the present invention can include one or more of the features discussed herein. Further, while one or more examples can be discussed as having certain advantageous features, one or more of such features can also be used with the various examples of the invention discussed herein. In similar fashion, while exemplary examples can be discussed below as device, system, or method examples, it is to be understood that such exemplary examples can be implemented in various devices, systems, and methods of the present invention.

### BRIEF DESCRIPTION OF THE DRAWINGS

The accompanying drawings, which are incorporated in and constitute a part of this specification, illustrate multiple examples of the presently disclosed subject matter and serve to explain the principles of the presently disclosed subject matter. The drawings are not intended to limit the scope of the presently disclosed subject matter in any manner. In the drawings:

FIG. 1 is a side cross sectional view of an injector, according to aspects of the present disclosure.

FIGS. 2A and 2B are views of an inner syringe barrel (see FIG. 2A) and a diluent ejector and powder barrel (see FIG. 2B), according to aspects of the present disclosure.

FIG. 2C is a cross sectional view of the distal end of an assembled syringe barrel, according to aspects of the present disclosure.

FIG. 3 is a cross sectional schematic showing a crown of thorns, according to aspects of the present disclosure.

FIG. 4A shows a latch of an injector, according to aspects of the present disclosure.

FIG. 4B shows a latch being inserted into a syringe barrel, according to aspects of the present disclosure.

FIG. 5 shows a needle and needle hub being inserted into a syringe barrel, according to aspects of the present disclosure.

FIG. 6A shows a needle cap being assembled over a syringe barrel, according to aspects of the present disclosure. FIG. 6B is a detailed view of a proximal end of the needle cap, according to aspects of the present disclosure.

FIG. 7A shows a needle cap assembled over a syringe barrel, according to aspects of the present disclosure. FIG. 7B is a detailed view of a track for the needle cap, according to aspects of the present disclosure.

FIG. 8A is a detailed view of two powder barrels, the bottom barrel showing a cross sectional view thereof, according to aspects of the present disclosure. FIG. 8B shows the powder barrel being assembled within a syringe barrel, according to aspects of the present disclosure.

FIG. 9 shows a diluent being added to the syringe barrel shown in FIG. 8B, according to aspects of the present disclosure.

FIG. 10A shows a disassembled diluent injector and corresponding stopper, according to aspects of the present disclosure. FIG. 10B shows the diluent injector and corresponding stopper of FIG. 10A being placed into the syringe barrel of FIG. 9, according to aspects of the present disclosure.

FIG. 11 shows the diluent injector and corresponding stopper assembled into the syringe barrel of FIG. 10B, according to aspects of the present disclosure.

FIG. 12 shows a spring container and a spring release, according to aspects of the present disclosure.

FIG. 13A is a detailed view of a spring container engaged with a spring release; FIG. 13B is elevation view of the spring container engaged with the spring release, according to aspects of the present disclosure.

FIG. 14 shows the spring release of FIG. 13B being inserted into the partially-assembled injector shown in FIG. 11, according to aspects of the present disclosure.

FIG. 15 shows a mixing spring being added to the injector shown in FIG. 14, according to aspects of the present disclosure.

FIG. 16 is perspective view of a distal end of an injector, showing a mixing spring added to the end of the injector, and showing a release ring of the spring release engaged with a spring container, according to aspects of the present disclosure.

FIG. 17A is a distal end perspective view of the mixing spring of FIG. 15 inserted into the syringe barrel; FIG. 17B is a side view of an injector showing a retaining clip being added to cap off the mixing spring, according to aspects of the present disclosure.

FIG. 18 is a cross sectional view of the distal end of an injector after the addition of the retaining clip of FIG. 17B, according to aspects of the present disclosure.

FIG. 19 is a side elevational view of a housing and outer cap, according to aspects of the present disclosure.

FIG. 20 shows the assembled syringe barrel of FIG. 18 being inserted into the housing of FIG. 19, according to aspects of the present disclosure.

FIG. 21 is a side elevational view of an assembled injector, according to aspects of the present disclosure.

FIGS. 22A-22C show the rotation of an outer cap to actuate an injector, according to aspects of the present disclosure. FIG. 22A shows the injector at an initial state, FIG. 22B shows the injector with a twisted cap to activate reconstitution, and FIG. 22C shows the injector with the cap removed.

FIGS. 23A and 23B are partially transparent views of an injector, according to aspects of the present disclosure. FIG. 23A shows the outer cap on the injector, and FIG. 23B shows the outer cap removed so the injector is ready for injection.

FIGS. 24A-24C are cross-sectional views of a mixing chamber, and the views display the procession of a latch as it is released from engagement with a needle hub in order to deliver the reconstituted active pharmaceutical agent, according to aspects of the present disclosure. FIG. 24A shows a position of the latch during reconstitution, FIG. 24B shows the latch released from a needle hub and progressing distally during an injection, and FIG. 24C shows the latch when the injection has been completed.

FIGS. 25A and 25B show needle cap lockout features, according to aspects of the present disclosure. FIG. 25A shows a barrel pin in a resting slot (before injection), and FIG. 25B shows the barrel pin in a lockout slot (after injection).

FIGS. 26A and 26B show alternative designs for the injector, according to aspects of the present disclosure. FIG. 26A shows a syringe barrel with only a single distal stopper and no powder barrel. FIG. 26B shows a device with no powder barrel or corresponding powder barrel stopper, and with only a first and second tearable seal.

FIG. 27 is a flowchart showing a method of administering an active pharmaceutical agent to a subject through an injector, according to aspects of the present disclosure.

FIG. 28 is a flowchart showing at least a portion of a method of assembling an injector, according to aspects of the present disclosure.

FIG. 29 is a flowchart showing at least a portion of a method of assembling an injector, according to aspects of the present disclosure.

FIG. 30 is a flowchart showing at least a portion of a method of assembling an injector, according to aspects of the present disclosure.

FIG. 31 is a flowchart showing a method of manufacturing an active pharmaceutical agent, according to aspects of the present disclosure.

FIG. 32 is a table showing results of testing a spray dried hydrocortisone sodium succinate as prepared using the method of FIG. 31.

FIG. 33 is a table showing example spray-drying conditions for hydrocortisone sodium succinate, according to aspects of the present disclosure.

### DETAILED DESCRIPTION

Specific examples of the present invention are now described in detail with reference to the Figures, where identical reference numbers indicate elements which are functionally similar or identical. The examples provide solutions for drug injectors and injector systems that provide simplified, user-friendly, and safe features. The described injectors and injector systems include reconstitution devices that enable one-step activation and reconstitution. Active pharmaceutical agents are maintained separately from diluent, and combination can be completed by the proximal movement of a diluent ejector through the system. The features of the injectors described herein can also include lockout features to prevent unintended reuse of spent devices.

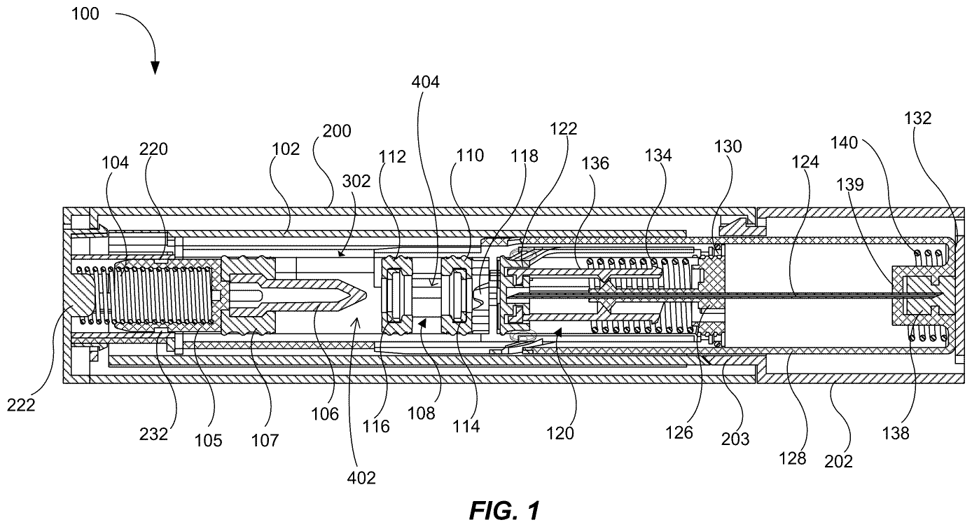

Referring now to FIG. 1, the figure provides an example cross sectional view of an injector 100, according to aspects of the present disclosure. The features of the injector 100 can be discussed in terms of the progression of activating the device. The injector 100 can be activated by twisting an outer cap 202 in relation to an outer housing 200. The twisting can activate the mixing of an active pharmaceutical agent 404 with a diluent 402, thereby “reconstituting” the active pharmaceutical agent 404. The outer cap 202 can engage with a spring release 102 positioned distal to the outer cap 202, and twisting (or turning) of the outer cap 202 can cause the spring release 102 to also turn within the housing 200. To clarify, when reference is made to “proximal” or “distal” in this disclosure, it will be understood to be from the point of view of a person self-administering an injection using the injector 100. For example, the parts of the injector 100 closest to the person are “proximal,” and the parts farthest from the person are “distal.” In FIG. 1, therefore, the parts to the right in the figure are “proximal,” as that is the direction of the skin of the person self-administering the injection.

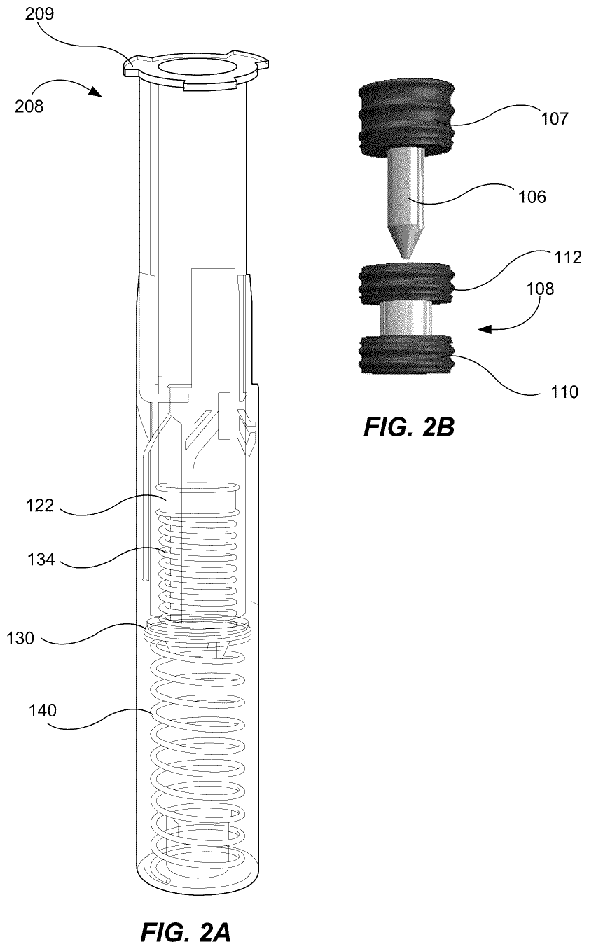

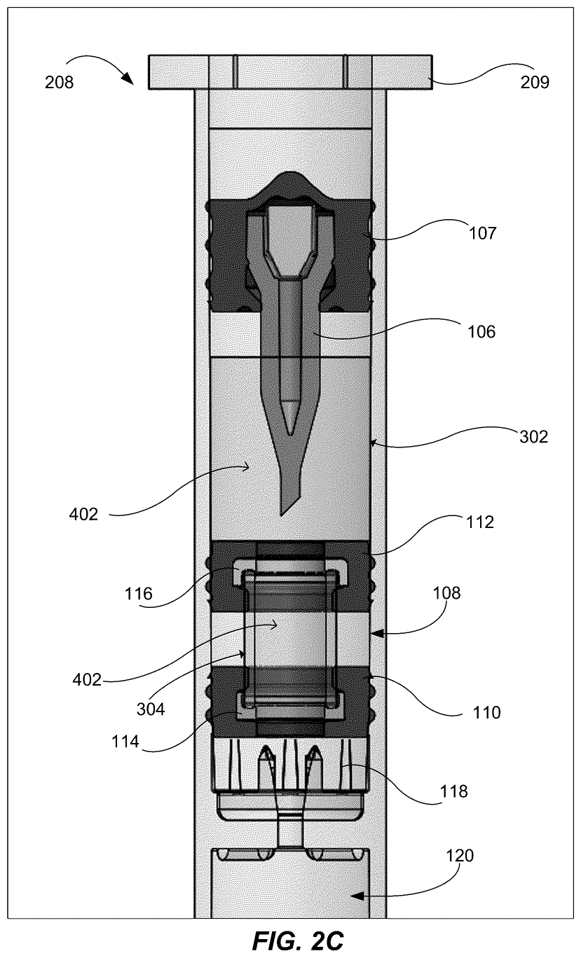

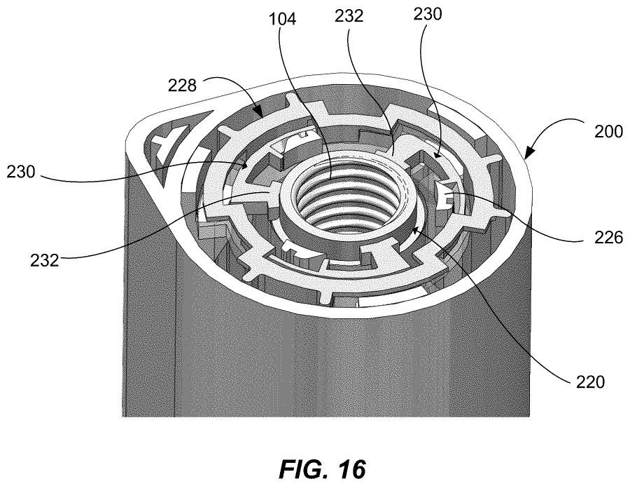

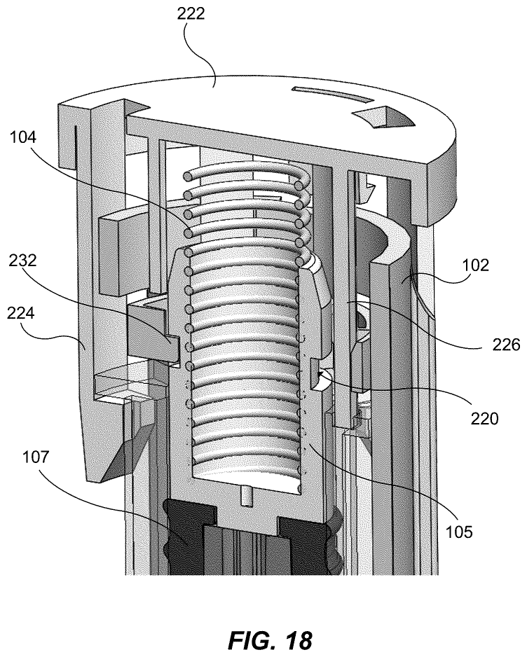

Referring now again to the spring release 102, actuation of the spring release 102 can cause a mixing spring 104 to release and move a diluent ejector 106 proximally through the injector 100. As can be seen in FIG. 1, the mixing spring 104 can be positioned within a spring container 105, and the spring container 105 can be releasably engageable with the spring release 102 (e.g., via a cam 232 of the spring release 102 engaging with a groove 220 in the spring container 105; see FIGS. 16 and 18). The diluent ejector 106 can comprise a stopper 107. The injector 100 can also include a powder barrel 108 that houses an active pharmaceutical agent 404. The powder barrel 108 can include a proximal stopper 110 and a distal stopper 112. The proximal stopper 110 and the distal stopper 112 provide a fluid-tight seal between them and a syringe barrel 208 (see, e.g., FIG. 2A), such that fluid must pass through the powder barrel 108. The distal stopper 112 can include a first tearable seal 116, and the proximal stopper 110 can include a second tearable seal 114. The active pharmaceutical agent 404 is contained between the first tearable seal 116 and the second tearable seal 114. A fluid chamber 302 is defined between the first tearable seal 116 and the stopper 107 of the diluent ejector 106, and the diluent 402 is contained in this fluid chamber 302. The tearable seals 114, 116 can be a foil material, for example a metallic foil material, as the foil can easily be pierced during operation of the device. Other materials that can be pierced are contemplated.

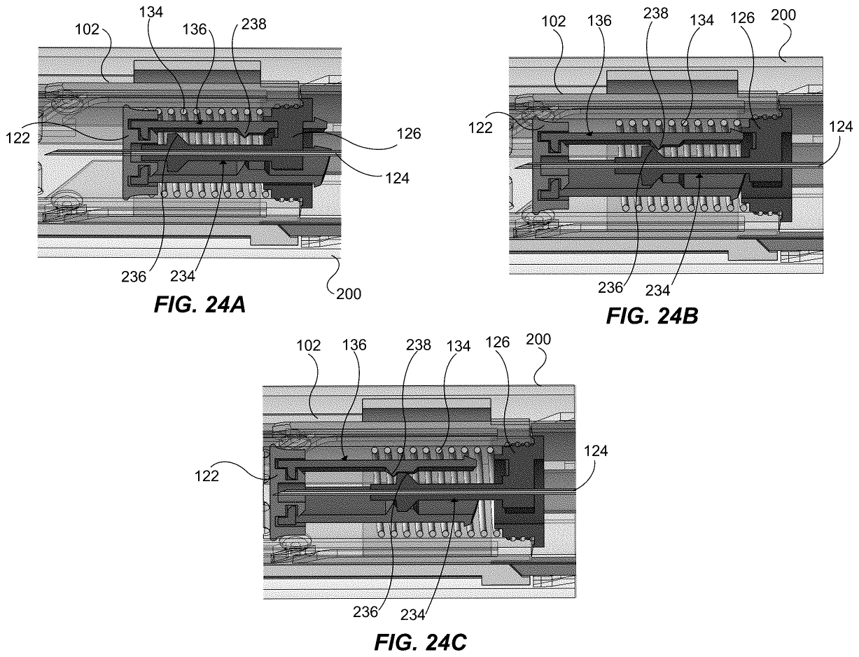

As the mixing spring 104 releases its compression to provide a bias to the diluent ejector 106 to drive the ejector proximally, the proximal tip of the diluent ejector 106 will pierce through the first tearable seal 116. At this point, the diluent 402 in the fluid chamber 302 can flow proximally and into the powder barrel 108. The positive pressure created in the fluid chamber 302 as a result of the proximal movement of the fluid and the diluent ejector 106 causes the powder barrel 108 to also move proximally and towards a crown of thorns 118. The crown of thorns 118 will then pierce the second tearable seal 116, and the diluent 402 and the partially-reconstituted active pharmaceutical agent 404 will flow through the powder barrel 108, through the crown of thorns 118, and toward a mixing-chamber seal 122. The mixing-chamber seal 122 provides a fluid-tight seal between it and the syringe barrel 208 (see, e.g., FIG. 2A), such that fluid cannot pass proximally through the mixing-chamber seal 122 unless the seal is pierced (as will be defined below and shown in more detail in FIGS. 24A-24C).

As the pressure and the diluent ejector 106 continues to cause fluid to move proximally, the mixing-chamber seal 122 will be pushed proximally, and in doing so it will move to form a mixing chamber 120 in which to allow the active pharmaceutical agent 404 to fully reconstitute. The movement of the mixing-chamber seal 122 proximally causes an injection spring 134 to collapse under the positive pressure. The mixing-chamber seal 122 also includes a latch 136 extending therefrom that is positioned to engage with a needle hub 126 proximal to the latch 136. A needle cap seal 130 seals the area between the needle hub 126 and the needle cap 128. A needle 124 is fixed within the needle hub 126, and the needle 124 does not translate within the injector 100 as the mixing-chamber seal 122 moves proximally. As such, a distal end of the needle 124 will pierce the mixing-chamber seal 122 as the mixing-chamber seal 122 moves proximally. When the mixing-chamber seal 122 has sufficiently moved proximally, the latch 136 will engage and connect with the needle hub 126. Once the latch 136 has engaged with the needle hub 126, the mixing chamber 120 can be considered fully formed, allowing the active pharmaceutical agent 404 to fully reconstitute. After reconstitution, the injector 100 is ready for injection.

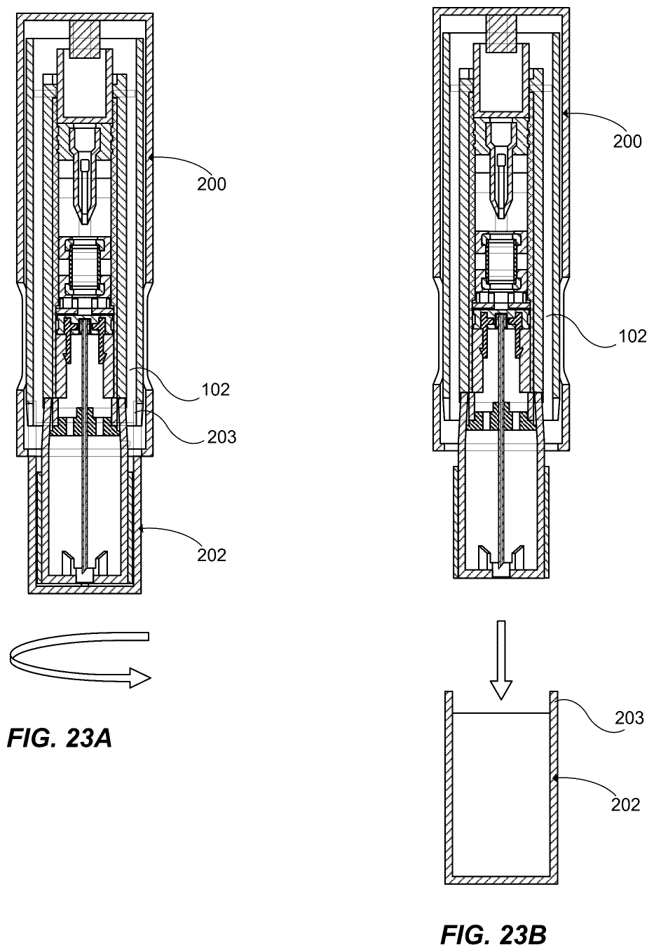

The outer cap 202 can be removed (see FIG. 23B), and a proximal end 132 of a needle cap 128 can be placed at the patient's skin. The proximal end 132 of the needle cap 128 can include a needle stopper seal 138 that can be pierced by the needle 124. As shown, the needle 124 can be at rest within the needle stopper seal 138 when the injector 100 is in an unfired configuration. The operator of the injector 100 can then move the housing 200 toward the skin, and in doing so the needle cap 128 will move distally with respect to the housing 200, thereby unsheathing the distal end of the needle 124. A platform 139 at the distal end of the needle cap 128 can disengage the latch 136 from the needle hub 126 to start the injection process. The injection spring 134 decompresses, and a positive pressure is again created within the mixing chamber 120, and the pressure can be alleviated by the reconstituted active pharmaceutical agent 404 flowing into the distal end of the needle 124, through the needle 124, and into the patient.

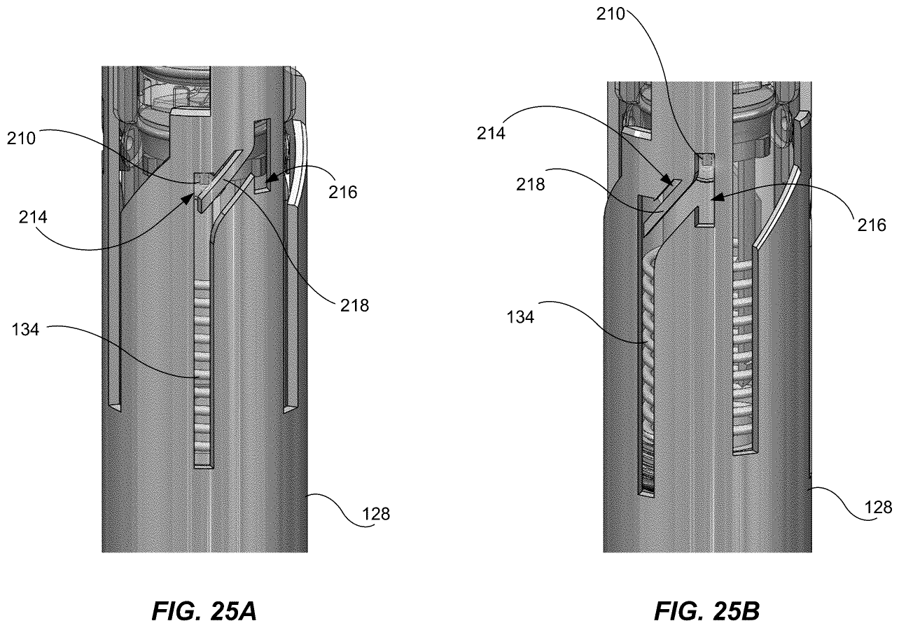

Once the reconstituted active pharmaceutical agent 404 has been administered, the user of the injector 100 can retract the needle from the patient's skin, and the needle 124 can be re-sheathed by the needle cap 128. To do so, a needle cap spring 140 can expand and force the proximal end 132 of the needle cap 128 back over the needle 124. As will be described in greater detail in FIGS. 25A and 25B, a lockout mechanism can be provided to ensure the spent injector 100 cannot be reused.

FIGS. 2A and 2B are views of an inner syringe barrel 208 (see FIG. 2A) and a diluent ejector 106 and powder barrel 108 (see FIG. 2B), according to aspects of the present disclosure. In FIG. 2A, the inner syringe barrel 208 is transparent to show locations of, for example, the mixing-chamber seal 122, the injection spring 134, the needle cap seal 130, and the needle cap spring 140. The inner syringe barrel 208 can be positioned within the housing 200 (see FIG. 1), and the feature creates the fluid channels for the diluent 402 and active pharmaceutical agent 404. The distal end (i.e., top in FIG. 2A) of the inner syringe barrel 208 can include a barrel lip 209 that can interact with a retaining clip 222 (see FIG. 18). FIG. 2B shows the diluent ejector 106 with its respective stopper 107, and the powder barrel 108 with its respective proximal stopper 110 and distal stopper 112. FIG. 2C is a cross sectional view of the distal end of an assembled syringe barrel 208, according to aspects of the present disclosure. The view shows locations of the diluent ejector 106, the respective chambers (i.e., fluid chamber 302, powder chamber 304, and mixing chamber 120), and additional features of the assembled, unfired syringe barrel 208 as described herein.

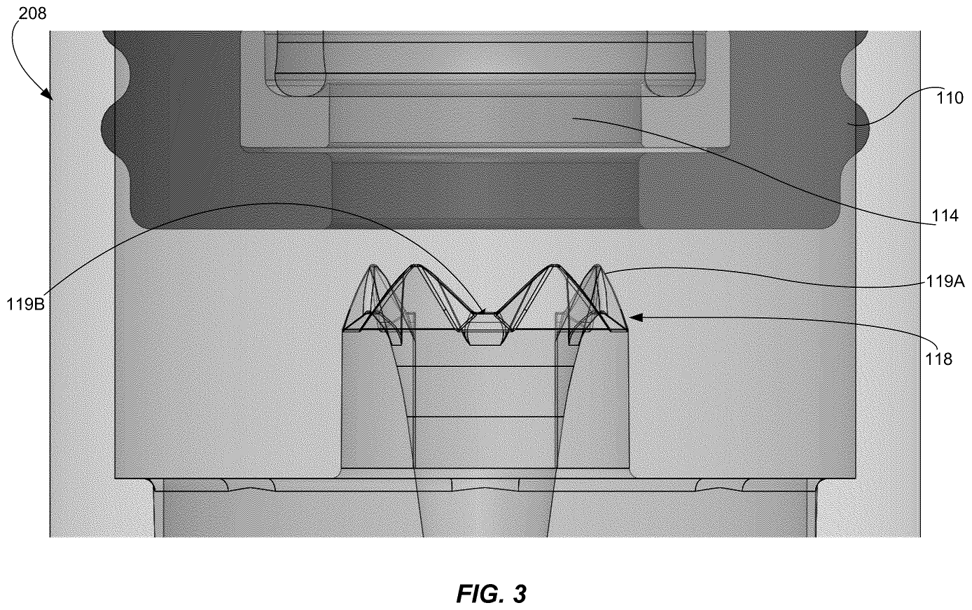

FIG. 3 is a cross sectional schematic showing the crown of thorns 118, according to aspects of the present disclosure. The crown of thorns 118 in any embodiment provides a sharp end distally in order to puncture or tear second tearable seal 114 when the powder barrel 108 translates proximally. As can be seen in FIG. 3, in one example the crown of thorns 118 can include one or more protrusion 119A that extend from inside the syringe barrel 208. These protrusions can extend from the material of the syringe barrel 208 and toward the second tearable seal 114. Between the protrusion(s) 119A is a fluid channel 119B that enables the at-least-partially reconstituted active pharmaceutical agent 404 from the powder barrel 108 to pass through to enter the mixing chamber 120. It will be appreciated, however, that the crown of thorns 118 described in this disclosure could be a single protrusion that punctures or tears second tearable seal 114, and nothing limits the crown of thorns 118 to look like the jagged feature shown in FIG. 3. In other words, the term “crown of thorns” is not intended to imply a structural limitation that it is fully circular like that shown in FIG. 3, but is intended only to indicate that the one or more protrusions extend distally from the location of the crown of thorns to puncture or pierce the second tearable seal 114.

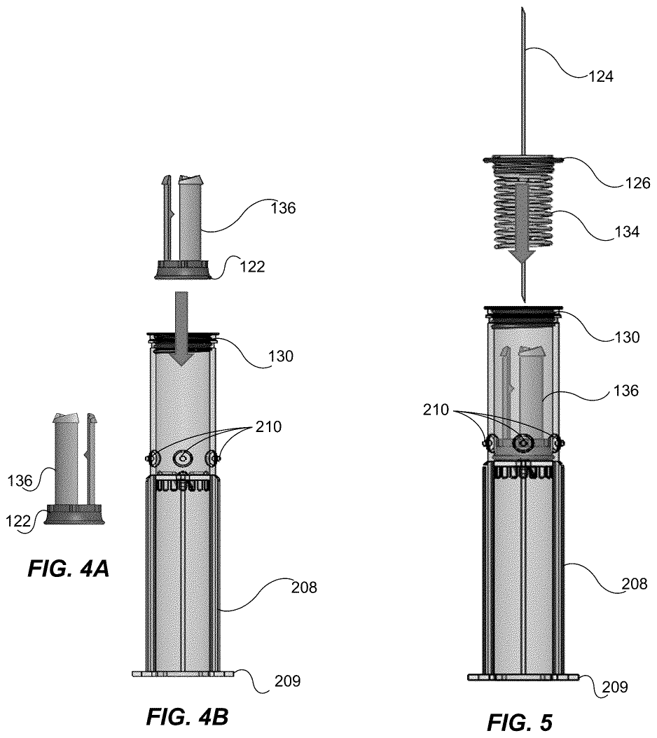

FIG. 4A shows a latch 136 extending from a mixing-chamber seal 122. FIG. 4B shows the latch 136 and mixing-chamber seal 122 being inserted into the syringe barrel 208. FIG. 5 shows the addition of the needle 124, needle hub 126, and injection spring 134 into the syringe barrel 208 after the latch 136 and mixing-chamber seal 122 have been inserted.

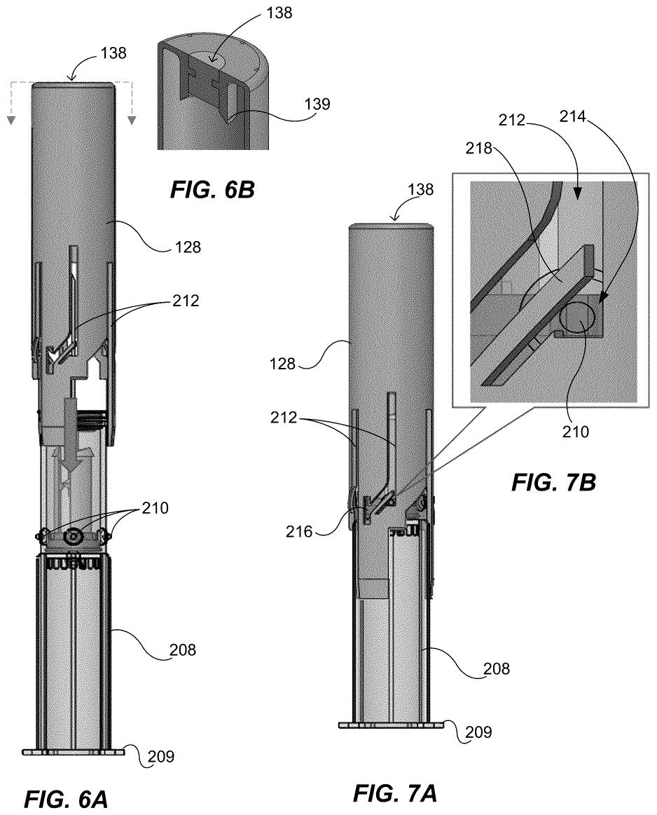

FIG. 6A shows a needle cap 128 being added over the partially-assembled syringe barrel 208 of FIG. 5; and FIG. 6B shows a detailed view of the needle stopper seal 138 at the proximal end 132 of the needle cap 128. The needle cap 128 can be slid down until one or more barrel pins 210 on the syringe barrel 208 slide into tracks 212 in the needle cap 128. FIG. 7A shows the needle cap 128 seated onto the syringe barrel 208, and the one or more barrel pins 210 are inserted into a resting slot 214. The needle cap 128 can also include the platform 139 extending distally therefore. More detail about the lockout mechanisms provided by the track 212 is described herein with respect to FIGS. 25A and 25B.

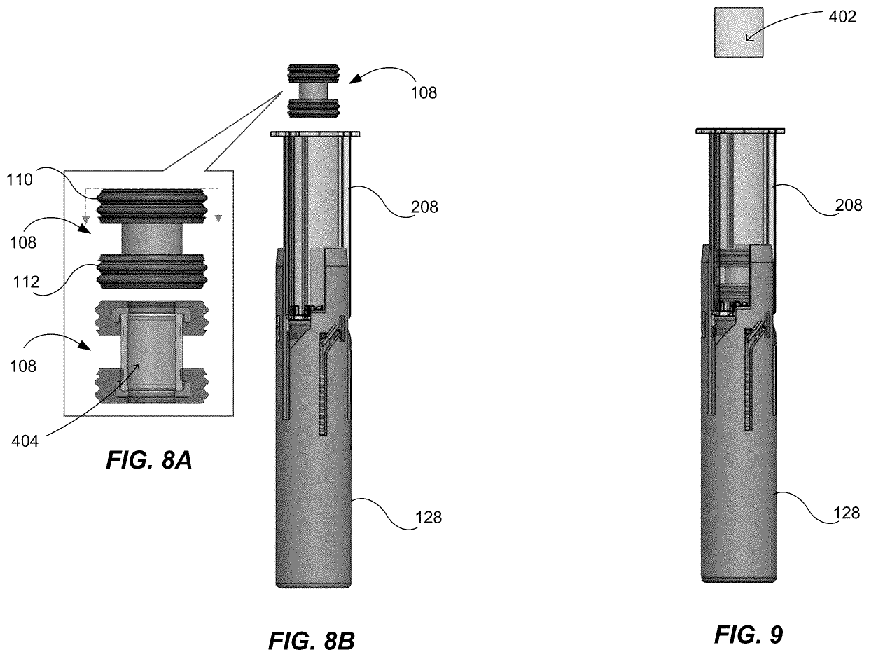

FIG. 8A is a detailed view of two powder barrels 108, the bottom powder barrel 108 showing a cross sectional view thereof, according to aspects of the present disclosure. FIG. 8B shows the powder barrel 108 being assembled within a syringe barrel 208, such as the syringe barrel 208 in FIG. 7A. As shown in FIG. 9, once the powder barrel 108 and the active pharmaceutical agent 404 therein are inserted into the syringe barrel 208, the distal stopper 112 and first tearable seal 116 provide one walled area within the syringe barrel 208, such that the diluent 402 can be added into the syringe barrel 208.

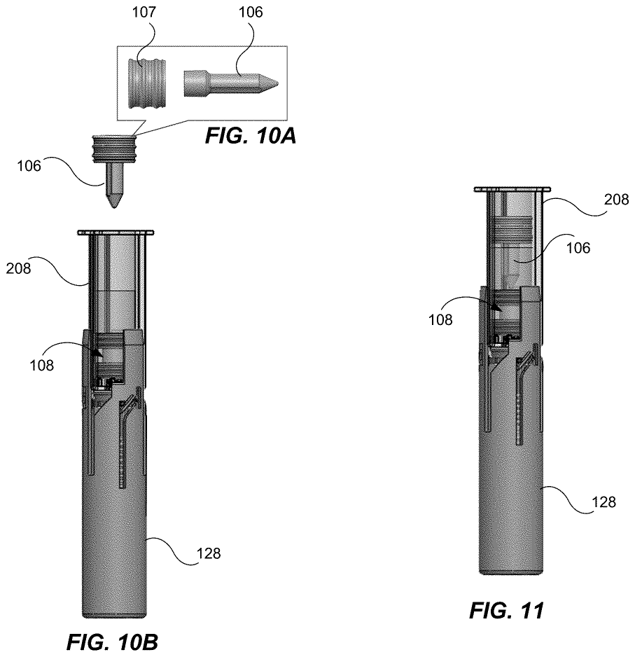

FIG. 10A shows an unassembled diluent ejector 106 and corresponding stopper 107. The stopper 107 can be made of an elastomeric material to form a fluid-tight seal around the moving diluent ejector 106, such that the diluent ejector 106, when moving proximally, pushes the diluent proximally. In FIG. 10B, the diluent ejector 106 and corresponding stopper 107 are inserted into the syringe barrel 208 to form the fluid chamber (see FIG. 1) and seal the diluent therein. FIG. 11 shows the injector after the diluent ejector 106 and corresponding stopper 107 has been inserted.

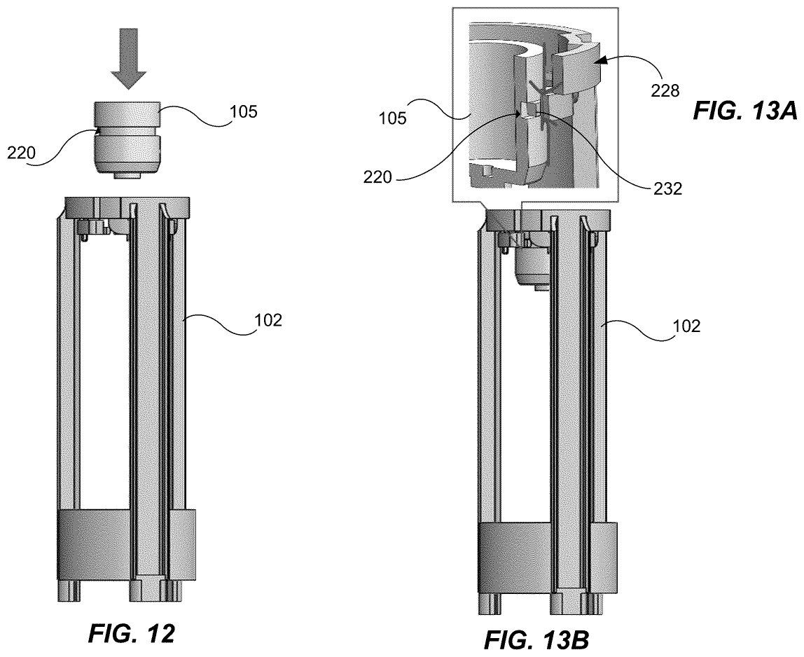

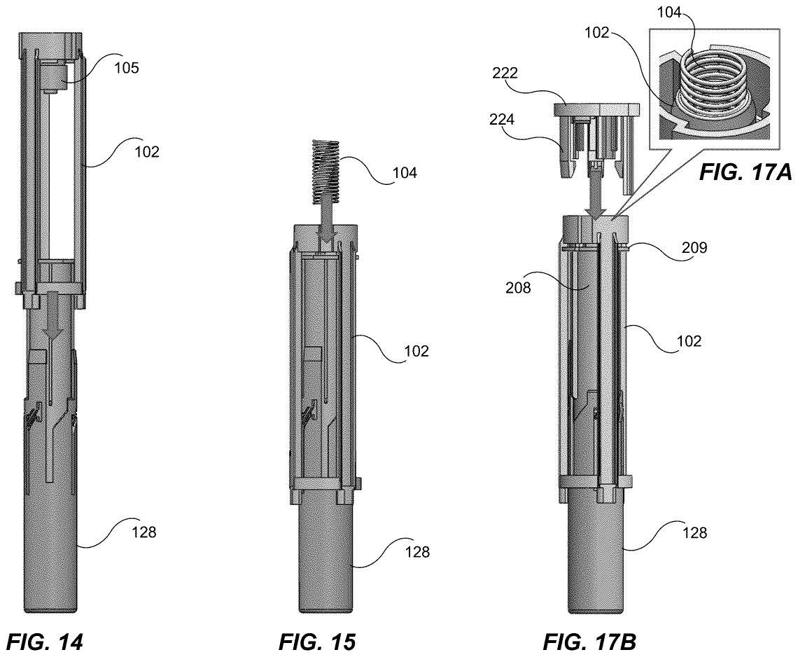

FIGS. 12-18 each highlight details of the spring release 102 mechanism described above. Referring now to FIG. 12, the spring container 105 can be engaged with the spring release 102 (see FIG. 16 for details on how a release ring 228 can be provided to engage with the spring container 105). The spring container 105 can be open at one end (the distal end) and closed at the other. FIG. 13A shows the engagement of (i) a cam 232 of the spring release 102 and (ii) the groove 220 on the spring container 105. As shown in FIG. 14, once the spring container 105 is engaged with the spring release 102, the combination of the two components can be assembled on to the partially-assembled injector 100 (e.g., the partially-assembled injector shown in FIG. 11). As stated above, the open end of the spring container 105 can be pointed distally such that the mixing spring 104 can be inserted into the open end, as shown in FIG. 15.

FIG. 16 shows the distal end of the injector 100 wherein the mixing spring 104 has been inserted, and the release ring 228 is engaged with the groove 220 of the spring container 105. It will be noted that the view in FIG. 16 is a partial cross-section, such that the distalmost portion of spring container 105 and mixing spring 104 are removed to more easily show engagement of the groove 220. The release ring 228 can comprise one or more spring arms 230. The example shown in FIG. 16 includes three spring arms 230 positioned around the circumference of the spring container 105, as three spring arms 230 is a sufficient number to ensure the spring container 105 is centered within the release ring 228. Each spring arm 230 includes a cam 232 that extends into the groove 220. The spring arms 230 are outwardly deflectable such that, once deflected, the cam 232 exits the groove 220 to release the spring container 105 to drive the diluent ejector 106 (see FIG. 1) proximally. This outward deflection of the spring arms 230 can be actuated by the twisting mechanism of the spring release 102. As stated above, the reconstitution can begin once the mixing spring 104 is released to drive the diluent ejector 106, and the twisting of the outer cap 202 in turn twists the spring release 102 to outwardly deflect the spring arms 230. It will also be appreciated that the amount of “twisting” required to activate the mixing can be adjusted by adjusting how much movement the spring release 102 needs to rotate in order to deflect the spring arms 230. For instance, the injector 100 can be configured such that outer cap 202 (and thus spring release 102) must be twisted 0° and 20° before the spring arms 230 have moved along the lever extension 226 sufficiently to release the spring container 102. This can be manipulated such that twisting the outer cap 202 includes twisting the outer cap 202 between 0° and 45° with respect to the housing 200 before the cam 232 is disengaged from the groove 220; such that twisting the outer cap 202 includes twisting the outer cap 202 between 0° and 90° with respect to the housing 200 before the cam 232 is disengaged from the groove 220; such that twisting the outer cap 202 includes twisting the outer cap 202 between 0° and 135° with respect to the housing 200 before the cam 232 is disengaged from the groove 220; such that twisting the outer cap 202 includes twisting the outer cap 202 between 0° and 180° with respect to the housing 200 before the cam 232 is disengaged from the groove 220; such that twisting the outer cap 202 includes twisting the outer cap 202 between 0° and 225° with respect to the housing 200 before the cam 232 is disengaged from the groove 220; such that twisting the outer cap 202 includes twisting the outer cap 202 between 0° and 270° with respect to the housing 200 before the cam 232 is disengaged from the groove 220; such that twisting the outer cap 202 includes twisting the outer cap 202 between 0° and 315° with respect to the housing 200 before the cam 232 is disengaged from the groove 220; such that twisting the outer cap 202 includes twisting the outer cap 202 between 0° and 360° with respect to the housing 200 before the cam 232 is disengaged from the groove 220; or such that twisting the outer cap 202 includes twisting the outer cap 202 greater than 360° with respect to the housing 200 before the cam 232 is disengaged from the groove 220.

Each spring arm 230 can be deflectable by a feature that acts as a ramp within the injector 100. In some examples, this ramp can be a lever extension extending, for example, from the distal end of the syringe barrel 208. In other examples, and as shown in FIG. 16, the ramp can be a lever extension 226 extending from a retaining clip 222 (see also FIG. 18). Referring now to FIG. 17A, the view shows the mixing spring 104 inserted into the spring container 105 prior to compressing the mixing spring 104. In FIG. 17B, a retaining clip 222 can be inserted at the distal end of the injector 100 to compress and contain the mixing spring 104. As shown, the retaining clip 222 can include connection extensions 224 extending therefrom that can engage with the barrel lip 209 to hold the mixing spring 104 compressed. FIG. 18 is a cross sectional view of the distal end of the injector 100 after the addition of the retaining clip 222 of FIG. 17B, according to aspects of the present disclosure. The figure provides a detailed view of the engagement of the connection extensions 224 with the barrel lip 209. Here, the cam 232 is also extending into the groove 220, and rotation of the spring release 102 causes the spring arms 230 to track along the lever extension 226 and deflect outwardly.

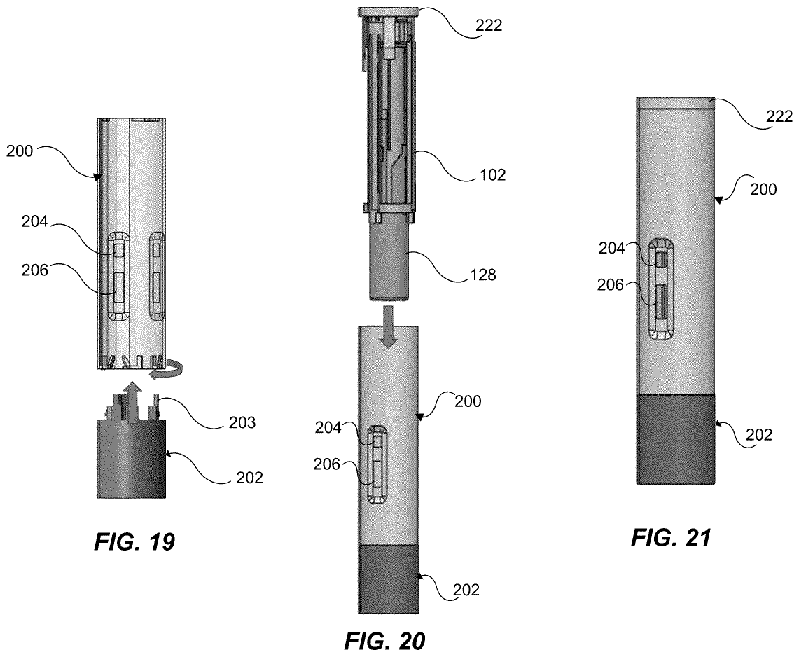

FIGS. 19-20 show the final assembly steps of the injector. FIG. 19 is a side elevational view of a housing 200 and outer cap 202, according to aspects of the present disclosure. The housing 200 and outer cap 202 can be combined prior to the insertion of the assembled syringe barrel 208. FIG. 20 shows the assembled syringe barrel 208 of FIG. 18 being inserted into the housing 200 of FIG. 19, according to aspects of the present disclosure. FIG. 21 is a side elevational view of the assembled injector 100, according to aspects of the present disclosure. FIGS. 19-20 also provide views of optional windows into the housing 200 that can provide visual information related to the state of reconstitution. The housing 200 can include a powder-inspection window 204 placed proximate a location of the powder barrel 108. The powder-inspection window 204 can provide a view of the powder barrel 108 before or during a first mixing of the active pharmaceutical agent and the diluent. For example, the powder-inspection window 204 can be located such that the active pharmaceutical agent 404 in the powder barrel 108 is visible before reconstitution, and once the diluent ejector 106 pierces the first tearable seal 116, the powder-inspection window 204 can show that this initial piercing has occurred. The housing 200 can also include a reconstitution window 206 placed within a wall of the housing 200 at a location proximal to the crown of thorns 118 (i.e., proximate the mixing chamber 120. The reconstitution window 206 can provide a view within the injector 100 proximate a location of a final mixing of the active pharmaceutical agent 404 and the diluent 402. The windows 204, 206 can be translucent plastic materials, or the windows 204,206 can simply be openings in the wall of the housing 200.

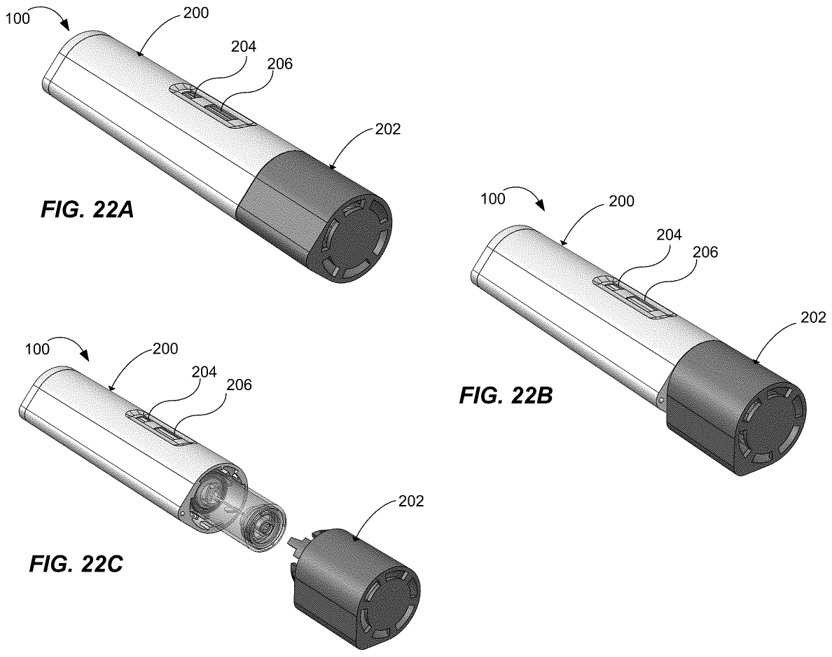

FIGS. 22A-22C shows the rotation of the outer cap 202 to actuate the reconstitution, according to aspects of the present disclosure. FIG. 22A shows the injector 100 at an initial state. FIG. 22B shows the injector 100 with a twisted outer cap 202 to such that the mixing spring 104 is actuated, as described above. FIG. 22C shows the injector 100 with the outer cap 202 removed. FIGS. 23A and 23B are partially transparent views of an injector 100 being primed by twisting the outer cap 202 and removing the outer cap 202.

FIGS. 24A-24C are cross-sectional views of a mixing chamber 120, and the views offer details of the procession of the latch 136 as it is released from engagement with the needle hub 126 in order to deliver the reconstituted active pharmaceutical agent 404 through the needle 124. As can be seen in FIG. 24A, at this point, the injector 100 is primed and ready for injection. The needle 124 now extends distal to the mixing-chamber seal 122, and as such the reconstituted active pharmaceutical agent 404 can be delivered through the needle by moving the mixing-chamber seal 122 distally again. To release the latch 136 from the needle hub 126, the platform 129 (see FIG. 6B) on the needle cap 128 can contact the proximal end of the latch 136 that extends through the needle hub 126. This causes the latch 136 to disengage, and the injection spring 134 will push the mixing-chamber seal 122 distally to eject the fluid from the needle 124. The injector can include a few feedback mechanisms during this delivery process. For example, the initial contact of the platform 129 and the latch 136 can provide audible and/or tactile feedback as the latch 136 is disengaged from the needle hub 126. In FIG. 24B, the mixing-chamber seal 122 is pushed distally, expelling more fluid. The other feedback mechanism can occur or activate toward the end of the injection. The needle hub 126 can include a hub extension 234 extending distally and including an extension ramp 236. The latch 136 can include a latch ramp 238 that can engage with the extension ramp 236. For instance, as the latch 136 is pushed distally with the mixing-chamber seal 122, the extension ramp 236 can abut the latch ramp 238 to provide audible and/or tactile feedback that the injection of the fluid is nearly complete. In FIG. 24C, the injection is complete.

FIGS. 25A and 25B show needle cap 128 lockout features, according to aspects of the present disclosure. FIG. 25A shows a barrel pin 210 in a resting slot 214, which is the configuration that the injector 100 can be in prior to injection. As the injection is performed and the needle cap 128 is pressed to the skin while the housing 200 is moved proximally, the barrel pin 210 will travel along the track 212 after passing by a deflector 218. After injection, the needle cap spring 140 (see FIG. 1) will push the needle cap 128 back out to sheath the needle 124, and in doing so the barrel pin 210 will track back through the track 212, the barrel pin 210 will contact the deflector 218, and the deflector 218 will cause the needle cap 128 to rotate such that the barrel pin 210 travels into lockout slot 216. In FIG. 25B, the barrel pin 210 is in the lockout slot 216, and the needle 124 remains sheathed so that the spent device is not inadvertently used again.

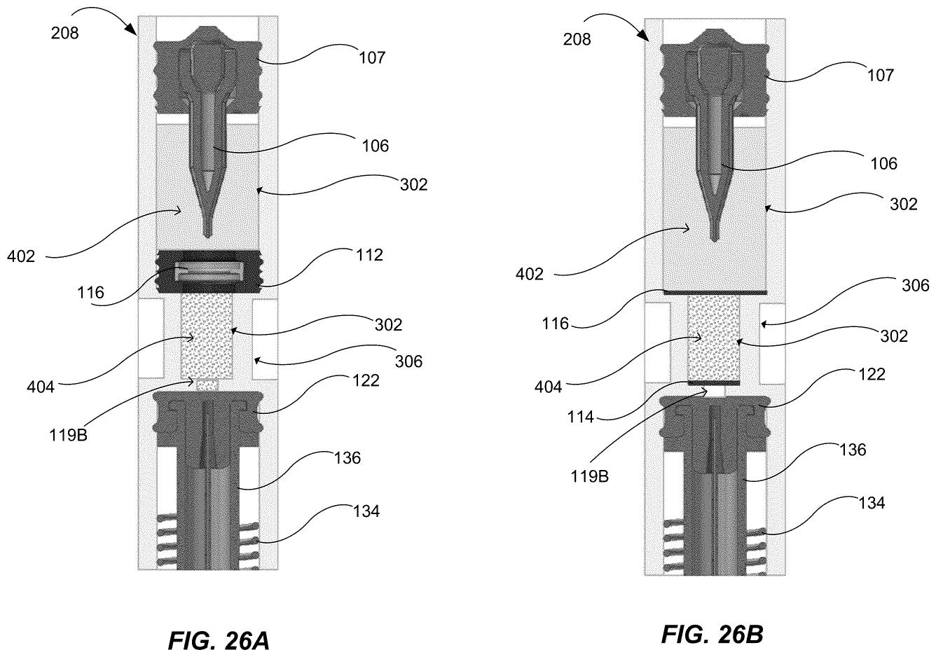

FIGS. 26A and 26B show alternative designs for the injector 100, according to aspects of the present disclosure. FIG. 26A shows a syringe barrel 208 with only a single distal stopper 112 and no powder barrel 108. In this example, there is no powder barrel to define a powder chamber 304, which was the case in the example shown in FIG. 1. In the example shown in FIG. 26A, the powder chamber 304 is defined at one end by a distal stopper 112 comprising a first tearable seal 116 and at the other end by the mixing-chamber seal 122 (which is slid distally in this resting, unfired position in the figure). A fluid channel 119B can extend between the powder chamber 304 and the mixing-chamber seal 122. In some examples, the syringe barrel 208 can have a narrowed section 306 around the powder chamber 304. In this example, when the diluent ejector 106 is fired proximally, the proximal tip of the diluent ejector 106 will pierce through the first tearable seal 116. At this point, the diluent 402 in the fluid chamber 302 can flow proximally and into the powder chamber 304. The positive pressure created in the powder chamber 304, as a result of the proximal movement of the fluid through the fluid channel 119B, causes the mixing-chamber seal 122 to move proximally. As such, the mixing chamber 120 will form and the reconstitution process can proceed as in the other examples outlined above.

FIG. 26B shows a syringe barrel 208 with no powder barrel 108 and without any corresponding stoppers between the diluent ejector 106 and the mixing-chamber seal 122. In this example, the powder chamber 304 is defined at one end by a first tearable seal 116 and at the other end by a second tearable seal 114. A fluid channel 119B can extend between the second tearable seal 114 and the mixing-chamber seal 122. In some examples, the syringe barrel 208 can have a narrowed section 306 around the powder chamber 304. In this example, when the diluent ejector 106 is fired proximally, the proximal tip of the diluent ejector 106 will pierce through the first tearable seal 116. At this point, the diluent 402 in the fluid chamber 302 can flow proximally and into the powder chamber 304. The positive pressure created in the powder chamber 304, as a result of the proximal movement of the fluid into the powder chamber 304, causes the second tearable seal 114 to tear. Then the at-least-partially mixed active pharmaceutical agent 404 will flow through fluid channel 119B and cause the mixing-chamber seal 122 to move proximally. As such, the mixing chamber 120 will form and the reconstitution process can proceed as in the other examples outlined above. In some alternative examples, the device shown in FIG. 26B may not include the second tearable seal 114, similar to what is shown in FIG. 26A. In this example, the powder chamber 304 will extend from the first tearable seal 116 to the mixing-chamber seal 122.

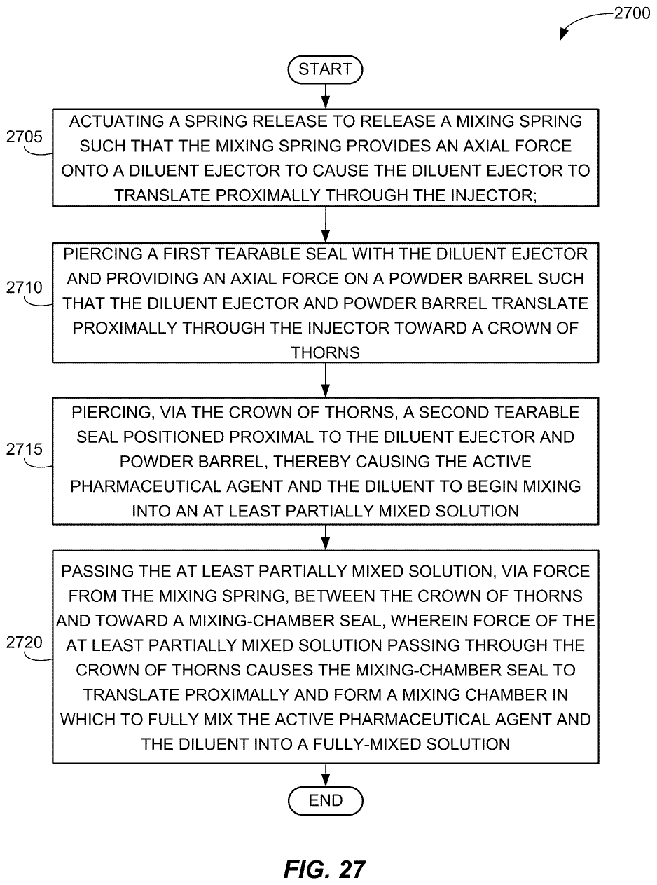

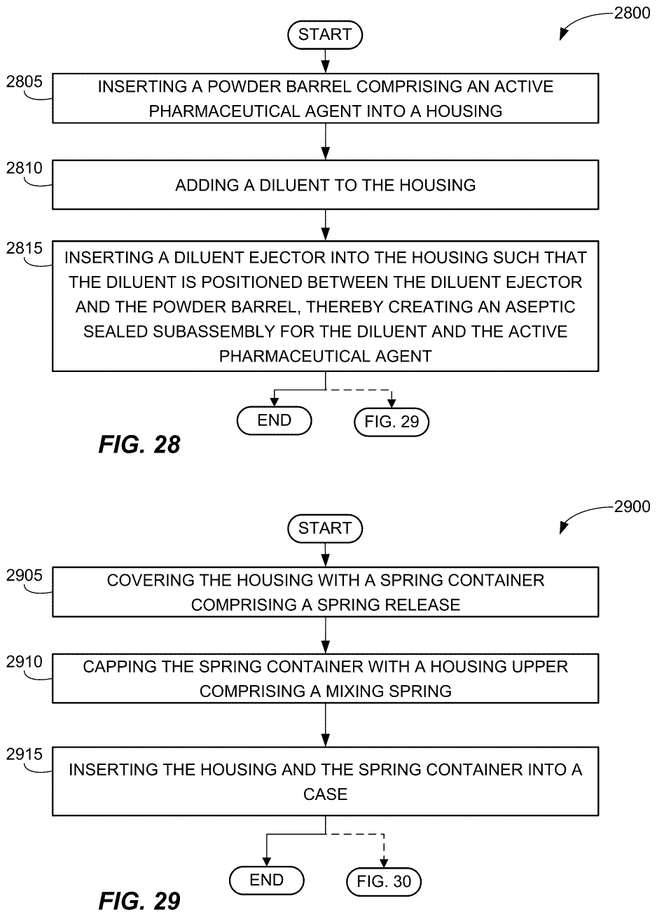

FIG. 27 is a flowchart showing a method 2700 of administering an active pharmaceutical agent to a subject through an injector 100, according to aspects of the present disclosure. Method 2700 can include actuating 2705 a spring release 102 to release a mixing spring 104 such that the mixing spring 104 provides an axial force onto a diluent ejector 106 to cause the diluent ejector 106 to translate proximally through the injector 100. Method 2700 can include piercing 2710 a first tearable seal 116 with the diluent ejector 106 and providing an axial force on a powder barrel 108 such that the diluent ejector 106 and powder barrel 108 translate proximally through the injector 100 toward a crown of thorns 118. Method 2700 can include piercing 2715, via the crown of thorns 118, a second tearable seal 114 positioned proximal to the diluent ejector 106 and powder barrel 108, thereby causing the active pharmaceutical agent 404 and the diluent 402 to begin mixing into an at least partially mixed solution. Method 2700 can include passing 2720 the at least partially mixed solution, via force from the mixing spring 104, between the crown of thorns 118 and toward a mixing-chamber seal 122, wherein force of the at least partially mixed solution passing through the crown of thorns 118 causes the mixing-chamber seal 122 to translate proximally and form a mixing chamber in which to fully mix the active pharmaceutical agent 404 and the diluent 402 into a fully-mixed solution (402+404). Method 2700 can end after the passing 2720 step or additional steps can be performed according to the embodiments described herein, FIG. 28 is a flowchart showing at least a portion of a method 2800 of assembling an injector 100, according to aspects of the present disclosure. Method 2800 can include inserting 2805 a powder barrel 108 comprising an active pharmaceutical agent 404 into a syringe barrel 208. Method 2800 can include adding 2810 a diluent 402 to the syringe barrel 208. Method 2800 can include inserting 2815 a diluent ejector 106 into the syringe barrel 208 such that the diluent is positioned between the diluent ejector 106 and the powder barrel 108, thereby creating an aseptic sealed subassembly for the diluent 402 and the active pharmaceutical agent 404. Method 2800 can end after the inserting 2815 step or additional steps can be performed according to the embodiments described herein. For example, the steps described in FIG. 28 can be performed in addition to those described for FIG. 28.

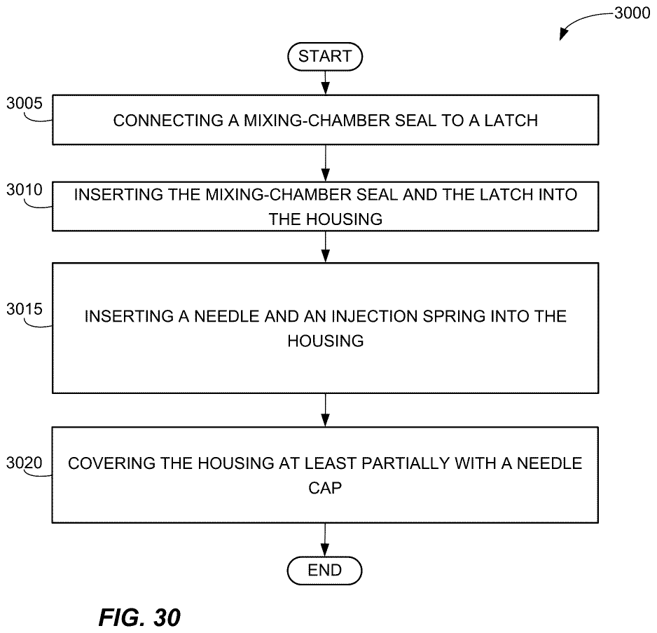

FIG. 29 is a flowchart showing at least a portion of a method 2900 of assembling an injector 100, according to aspects of the present disclosure. Method 2900 can include covering 2905 the syringe barrel 208 with a spring container 105 comprising a spring release 102. Method 2900 can include capping 2910 the spring container 105 with a retaining clip 222 comprising a mixing spring 104. Method 2900 can include inserting 2915 the syringe barrel 208 and the spring container 105 into a housing 200. Method 2900 can end after the inserting 2915 step or additional steps can be performed according to the embodiments described herein. For example, the steps described in FIG. 30 can be performed in addition to those described for FIG. 28 and/or FIG. 29.

FIG. 30 is a flowchart showing at least a portion of a method 3000 of assembling an injector 100, according to aspects of the present disclosure. Method 3000 can include connecting 3005 a mixing-chamber seal 122 to a latch 136. Method 3000 can include inserting 3010 the mixing-chamber seal 122 and the latch 136 into the syringe barrel 208. Method 3000 can include inserting 3015 a needle 124 and an injection spring 134 into the syringe barrel 208. Method 3000 can include covering 3020 the syringe barrel 208 at least partially with a needle cap 128. Method 3000 can end after the covering 3020 step or additional steps can be performed according to the embodiments described herein.

In any example described herein, the injector 100 can deliver, for example, a hydrocortisone sodium succinate sodium salt, hydrocortisone phosphate, and/or hydrocortisone acetate. However, as will be appreciated, other active agents can be included. In some examples, the active agents can be spray-dried or lyophilized, and that spray-dried or lyophilized product can be placed in the powder barrel 108. In certain examples, there can be advantages to spray drying the hydrocortisone sodium succinate sodium salt. Spray drying can be a simple, singular process from solution to powder, whereas lyophilization requires additional milling or micronization. Spray drying provides consistent particle size and flow properties, and there is a possibility for particle engineering to adjust size distribution if necessary. As such, this disclosure also provides solutions for spray drying hydrocortisone sodium succinate sodium salt to include in the powder barrel 108.

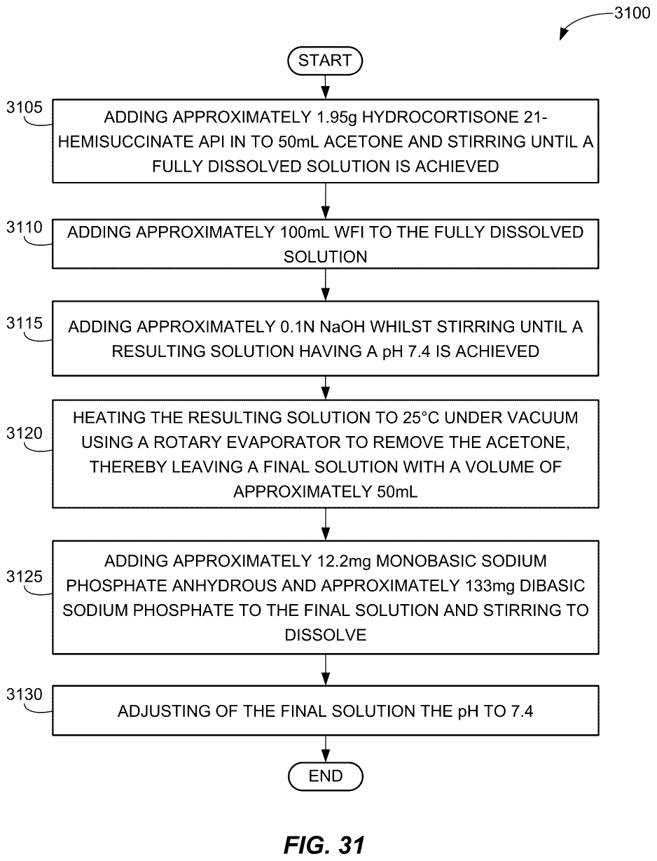

FIG. 31 is a flowchart showing a method 3100 of manufacturing an active pharmaceutical agent (e.g., active pharmaceutical agent 404), according to aspects of the present disclosure. Method 3100 can include adding 3105 approximately 1.95 g hydrocortisone 21-hemisuccinate API in to 50 mL acetone and stirring until a fully dissolved solution is achieved. Method 3100 can include adding 3110 approximately 100 mL WFI to the fully dissolved solution. Method 3100 can include adding 3115 approximately 0.1N NaOH whilst stirring until a resulting solution having a pH 7.4 is achieved. Method 3100 can include heating 3120 the resulting solution to 25 °C under vacuum using a rotary evaporator to remove the acetone, thereby leaving a final solution with a volume of approximately 50 mL. Method 300 can include adding 3125 approximately 12.2 mg monobasic sodium phosphate anhydrous and approximately 133 mg dibasic sodium phosphate to the final solution and stirring to dissolve. Method 3000 can include adjusting 3130 of the final solution the pH to 7.4. Method 3100 can end after the adjusting 3130 step, or additional steps can be performed as described herein or as will be appreciated.

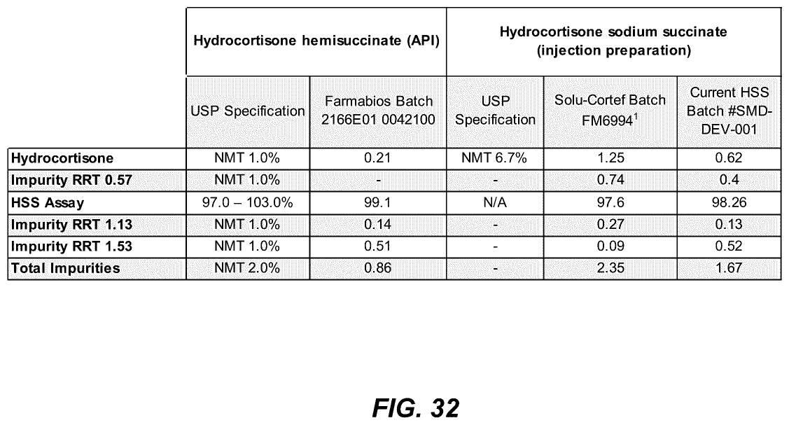

The steps outlined above were performed to identify the purity produced with such method 3100, and the data is provided in the table shown in FIG. 32. The tested spray-dried powder (labeled as “Current HSS” in the table) was analyzed by RP-HPLC and the purity/impurity profile compared to that of the API, hydrocortisone hemisuccinate and the RLD, Solu-Cortef™ measured in the same chromatographic system. The data (area %), with relevant United States Pharmacopeia (USP) specifications are shown in FIG. 32.

Compared to the feed API, the “Current HSS” shows increased levels of hydrocortisone (hydrolysis product) and an unknown impurity at RRT 0.57. The impurities seen at RRT 1.13 and RRT 1.53 are derived from the API and are not of concern since the API is supplied as GMP grade suitable for human use. The USP has specifications for hydrocortisone hemisuccinate of not more than 1% of any individual impurity and not more that 2% of total impurities. The API meets these specifications.

The USP also has specifications for hydrocortisone sodium succinate injection. The specifications allow for not more that 6.7% free hydrocortisone. The specifications do not address impurity levels in the HSS injection preparation. In conclusion, the “Current HSS” meets the required purity specifications.

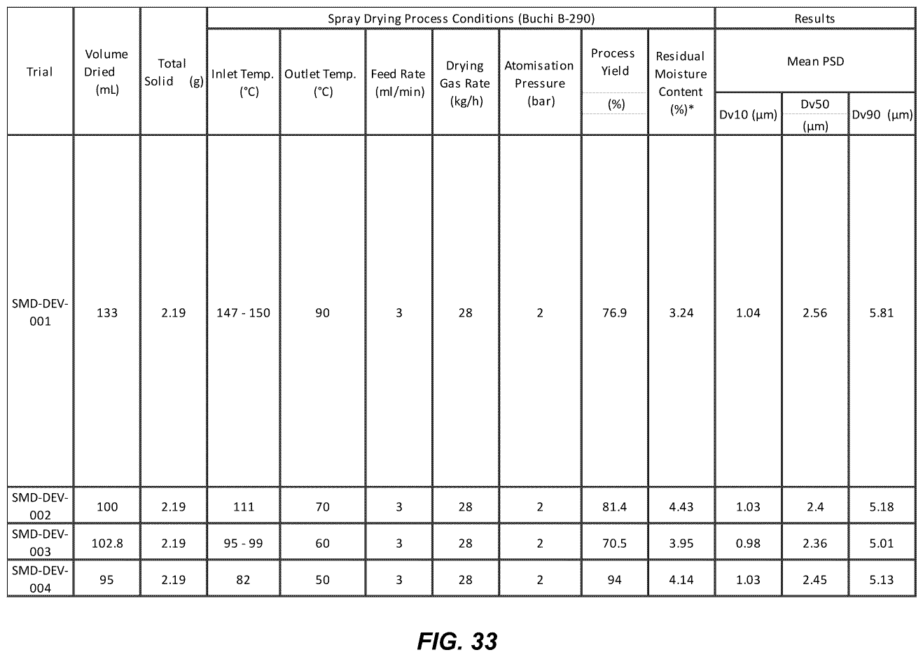

The final solution can be spray dried. Spray drying a therapeutic agent can be understood to mean a process of subjecting the therapeutic to high temperatures and then cooling the therapeutic, thereby providing an evaporative effect. In the instant case, the final solution of HSS can be spray dried according to the parameters shown in FIG. 33. For example, the HSS can be spray dried at an inlet temperature of from 82°C to 150 °C and with an outlet temperature of from 50 °C to 90 °C, e.g., with an inlet temperature of 147-150 °C and with an outlet temperature of 90 °C, with an inlet temperature of 111 °C and with an outlet temperature of 70 °C, with an inlet temperature of 95-99 °C and with an outlet temperature of 60 °C, with an inlet temperature of 82 °C and with an outlet temperature of 50 °C, etc. (see FIG. 33). The active pharmaceutical agent can be spray dried using nitrogen.

Examples of the present disclosure can be implemented according to at least the following clauses:
Clause 1: An injector for delivering a solution of an active pharmaceutical agent and a diluent to a subject, the injector comprising: a housing (200); a spring release (102); an outer cap (202) separably engaged with the spring release (102) and rotatable with the spring release (102); a diluent ejector (106) comprising a stopper (107); a mixing spring (104) adjacent the diluent ejector (106) providing a bias to the diluent ejector (106) within the housing (200); a powder barrel (108) comprising a first tearable seal (116) and a second tearable seal (114); a crown of thorns (118) positioned proximate the second tearable seal (114) and configured to pierce the second tearable seal (114) responsive to translation of the powder barrel (108) proximally within the housing (200); and a fluid chamber (302) disposed between the stopper (107) and the first tearable seal.
Clause 2: The injector of clause 1 further comprising a mixing-chamber seal (122) movable within the housing (200) responsive to a positive pressure caused by proximal translation of the diluent ejector (106) via the mixing spring (104).
Clause 3: The injector of clause 2 further comprising: a latch (136) connected to the mixing-chamber seal (122); and a needle hub (126) engageable with the latch (136)
Clause 4: The injector of clause 2 or 3 further comprising: a needle (124); a needle cap (128); and a needle cap spring (140).
Clause 5: The injector of clause 2, wherein the outer cap (202) is twistable with the spring release (102) from between 0° and 20°.
Clause 6: The injector of clause 2, wherein the outer cap (202) is twistable with the spring release (102) from between 0° and 45°.
Clause 7: The injector of clause 2, wherein the outer cap (202) is twistable with the spring release (102) from between 0° and 90°.
Clause 8: The injector of clause 2, wherein the outer cap (202) is twistable with the spring release (102) from between 0° and 135°.
Clause 9: The injector of clause 2, wherein the outer cap (202) is twistable with the spring release (102) from between 0° and 180°.
Clause 10: The injector of clause 2, wherein the outer cap (202) is twistable with the spring release (102) from between 0° and 225°.
Clause 11: The injector of clause 2, wherein the outer cap (202) is twistable with the spring release (102) from between 0° and 270°.
Clause 12: The injector of clause 2, wherein the outer cap (202) is twistable with the spring release (102) from between 0° and 315°.
Clause 13: The injector of clause 2, wherein the outer cap (202) is twistable with the spring release (102) from between 0° and 360°.
Clause 14: The injector of clause 2, wherein twisting the outer cap (202) comprises twisting the outer cap (202) greater than 360°.
Clause 15: The injector of any one of clauses 2 to 14 further comprising the active pharmaceutical agent (404) disposed within the powder barrel (108).
Clause 16: The injector of clause 15, wherein the active pharmaceutical agent (404) is a spray-dried powder stored in the powder barrel (108).
Clause 17: The injector of clause 16, wherein the active pharmaceutical agent (404) is a hydrocortisone sodium succinate sodium salt.
Clause 18: The injector of clause 16, wherein the powder barrel (108) comprises greater than 75 mg of the spray-dried powder.
Clause 19: The injector of any one of the preceding clauses, wherein the outer cap (202) is engageable with the housing (200) to cover and protect a needle cap (128).
Clause 20: The injector of any one of the preceding clauses, wherein a cross section of the housing (200) comprises a tear drop shape.
Clause 21: The injector of any one of the preceding clauses, wherein the housing (200) comprises a powder-inspection window (204) placed proximate a location of the powder barrel (108).
Clause 22: The injector of any one of the preceding clauses, wherein the housing (200) comprises a powder-inspection window (204) placed proximate a location of the powder barrel (108), the powder-inspection window (204) providing a view of the powder barrel (108) before or during a first mixing of the active pharmaceutical agent and the diluent.
Clause 23: The injector of any one of the preceding clauses, wherein the housing (200) comprises a reconstitution window (206) placed within a wall of the housing (200) and proximal to the crown of thorns (118), the reconstitution window (206) providing a view within the injector proximate a location of a final mixing of the active pharmaceutical agent and the diluent.
Clause 24: The injector of clause 23, wherein the housing (200) further comprises a powder-inspection window (204) placed proximate a location of the powder barrel (108), the powder-inspection window (204) providing a view of the powder barrel (108) before or during a first mixing of the active pharmaceutical agent and the diluent.
Clause 25: The injector of any of the preceding clauses further comprising a spring container (105) proximate the diluent ejector (106) and engaged with the mixing spring (104), the spring container (105) releasably engageable with the spring release (102), wherein disengagement of the spring container (105) from the spring release (102) releases the bias of the spring container (105) to actuate the diluent ejector (106).
Clause 26: The injector of clause 25, wherein: the spring container (105) comprises a circumferential groove (220); the spring release (102) comprises a spring arm (230) with a cam (232) extending therefrom, the cam (232) extending into the circumferential groove (220); and the spring arm (230) is configured to deflect radially to cause the cam (232) to disengage from the circumferential groove (220).
Clause 27: The injector of clause 26 further comprising a retaining clip (222) engaged with the housing (200) distal to the mixing spring (104), the retaining clip (222) comprising a lever extension (226) extending proximally and into the housing (200).
Clause 28: The injector of clause 27, wherein the lever extension (226) is positioned proximate the spring arm (230) and is engageable with the spring arm (230), wherein the spring arm (230) is configured to engage with the lever extension (226) in response to rotation of the spring release (102) thereby causing radial deflection of the spring arm (230).
Clause 29: A method of administering an active pharmaceutical agent to a subject through an injector, comprising: twisting an outer cap of the injector to activate mixing of the active pharmaceutical agent with a diluent, activating mixing comprising the steps of: actuating a spring release to release a mixing spring such that the mixing spring provides an axial force onto a diluent ejector to cause the diluent ejector to translate proximally through the injector; piercing a first tearable seal with the diluent ejector and providing an axial force on a powder barrel such that the diluent ejector and powder barrel translate proximally through the injector toward a crown of thorns; piercing, via the crown of thorns, a second tearable seal positioned proximal to the diluent ejector and powder barrel, thereby causing the active pharmaceutical agent and the diluent to begin mixing into an at least partially mixed solution; and passing the at least partially mixed solution, via force from the mixing spring, between the crown of thorns and toward a mixing-chamber seal, wherein force of the at least partially mixed solution passing through the crown of thorns causes the mixing-chamber seal to translate proximally and form a mixing chamber in which to fully mix the active pharmaceutical agent and the diluent into a fully-mixed solution.
Clause 30: The method of clause 29 further comprising priming the fully-mixed solution for injection, wherein priming comprises the steps of: piercing the mixing-chamber seal with a needle in response to the mixing-chamber seal translating proximally; and translating, via force from the mixing spring upon the active pharmaceutical agent and the diluent, a latch affixed to the mixing-chamber seal proximally toward a needle hub, wherein proximal translation of the mixing-chamber seal and latch load an injection spring.
Clause 31: The method of clause 30, wherein priming further comprises the step of deflecting the latch such that it engages with the deflects to secure the needle hub, thereby holding the injection spring in a compressed position.
Clause 32: The method of clause 30 or 31, further comprising: removing the outer cap from the injector; and injecting the fully-mixed solution, the injecting comprising the step of: placing a proximal end of a needle cap at the skin of the subject and moving a housing of the injector proximally such that the needle cap translates distally with respect to the housing and independently from the needle such that a proximal end of the needle exits the needle cap proximally; in response to the needle cap translating distally, causing the needle hub to translate distally within the injector to create a pressure in the fully-mixed solution between the needle hub and the diluent ejector; and delivering, via the pressure, the fully-mixed solution through the needle.
Clause 33: The method of clause 32, further comprising: removing needle from the skin by pulling the injector away from the subject; and causing the needle to be sheathed by the needle cap via a needle cap spring.
Clause 34: The method of clause 33, further comprising causing, via the needle cap spring, the needle cap to sheathe the needle permanently.
Clause 35: The method of any one of clauses 32 to 34, wherein delivering the fully-mixed solution comprises delivering approximately 1 mL of the fully-mixed solution.
Clause 36: The method of any one of clauses 32 to 35, wherein delivering the fully-mixed solution comprises delivering less than 1 mL of the fully-mixed solution.
Clause 37: The method of any one of clauses 32 to 36, wherein delivering the fully-mixed solution comprises delivering approximately 2 mL of the fully-mixed solution.
Clause 38: The method of any one of clauses 32 to 37, wherein delivering the fully-mixed solution comprises delivering less than 2 mL of the fully-mixed solution.
Clause 39: The method of any one of clauses 32 to 38, wherein delivering the fully-mixed solution comprises delivering greater than 2 mL of the fully-mixed solution.
Clause 40: The method of any one of clauses 32 to 39, wherein delivering the fully-mixed solution can be completed at any angle with respect to horizontal.
Clause 41: The method of any one of clauses 29 to 40, wherein the active pharmaceutical agent is a spray-dried powder stored in the powder barrel.
Clause 42: The method of clause 41, wherein the active pharmaceutical agent is a hydrocortisone sodium succinate sodium salt.
Clause 43: The method of clause 41 or 42, wherein the powder barrel comprises greater than 75 mg of the spray-dried powder.
Clause 44: The method of any one of clauses 29 to 43, wherein twisting the outer cap comprises twisting the outer cap between 0° and 20° with respect to a housing.
Clause 45: The method of any one of clauses 29 to 43, wherein twisting the outer cap comprises twisting the outer cap between 0° and 45° with respect to a housing.
Clause 46: The method of any one of clauses 29 to 43, wherein twisting the outer cap comprises twisting the outer cap between 0° and 90° with respect to a housing.
Clause 47: The method of any one of clauses 29 to 43, wherein twisting the outer cap comprises twisting the outer cap between 0° and 135° with respect to a housing.
Clause 48: The method of any one of clauses 29 to 43, wherein twisting the outer cap comprises twisting the outer cap between 0° and 180° with respect to a housing.
Clause 49: The method of any one of clauses 29 to 43, wherein twisting the outer cap comprises twisting the outer cap between 0° and 225° with respect to a housing.
Clause 50: The method of any one of clauses 29 to 43, wherein twisting the outer cap comprises twisting the outer cap between 0° and 270° with respect to a housing.
Clause 51: The method of any one of clauses 29 to 43, wherein twisting the outer cap comprises twisting the outer cap between 0° and 315° with respect to a housing.
Clause 52: The method of any one of clauses 29 to 43, wherein twisting the outer cap comprises twisting the outer cap between 0° and 360° with respect to a housing.
Clause 53: The method of any one of clauses 29 to 43, wherein twisting the outer cap comprises twisting the outer cap greater than 360° with respect to a housing.
Clause 54: The method of any of clauses 29 to 52, wherein the active pharmaceutical agent and the diluent are automatically and fully mixed in the mixing chamber in less than or equal to 20 seconds.
Clause 55: The method of clause 32 further comprising providing an audible cue when the fully-mixed solution is fully delivered through the needle.
Clause 56: A method of assembling an injector, the method comprising: inserting a powder barrel comprising an active pharmaceutical agent into a syringe barrel; adding a diluent to the syringe barrel; and inserting a diluent ejector into the syringe barrel such that the diluent is positioned between the diluent ejector and the powder barrel, thereby creating an aseptic sealed subassembly for the diluent and the active pharmaceutical agent.
Clause 57: The method of clause 56 further comprising: covering the syringe barrel with a spring container comprising a spring release; capping the spring container with a mixing spring and a retainer clip; and inserting the syringe barrel and the spring container into a housing.
Clause 58: The method of clause 56 or 57 further comprising: connecting a mixing-chamber seal to a latch; inserting the mixing-chamber seal and the latch into the syringe barrel; inserting a needle and an injection spring into the syringe barrel; and covering the syringe barrel at least partially with a needle cap.
Clause 59: The method of any one of clauses 56 to 58 further comprising manufacturing the active pharmaceutical agent, wherein creating the active pharmaceutical agent comprises: adding approximately 1.95 g hydrocortisone 21-hemisuccinate API in to 50 mL acetone and stirring until a fully dissolved solution is achieved; adding approximately 100 mL WFI to the fully dissolved solution; adding approximately 0.1N NaOH whilst stirring until a resulting solution having a pH 7.4 is achieved; heating the resulting solution to 25 °C under vacuum using a rotary evaporator to remove the acetone, thereby leaving a final solution with a volume of approximately 50 ml; adding approximately 12.2 mg monobasic sodium phosphate anhydrous and approximately 133 mg dibasic sodium phosphate to the final solution and stirring to dissolve; and adjusting of the final solution the pH to 7.4.
Clause 60: The method of clause 59, wherein the step of manufacturing the active pharmaceutical agent further comprises spray drying the final solution using nitrogen.
Clause 61: The method of clause 60, further comprising spray drying the final solution at an inlet temperature of from 82 °C to 150 °C, and with an outlet temperature of from 50 °C to 90 °C.
Clause 62: A method of manufacturing an active pharmaceutical agent comprising:

adding approximately 1.95 g hydrocortisone 21-hemisuccinate API in to 50 mL acetone and stirring until a fully dissolved solution is achieved;
adding approximately 100 mL WFI to the fully dissolved solution;
adding approximately 0.1N NaOH whilst stirring until a resulting solution having a pH 7.4 is achieved;
heating the resulting solution to 25 °C under vacuum using a rotary evaporator to remove the acetone, thereby leaving a final solution with a volume of approximately 50 mL;
adding approximately 12.2 mg monobasic sodium phosphate anhydrous and approximately 133 mg dibasic sodium phosphate to the final solution and stirring to dissolve; and
adjusting of the final solution the pH to 7.4.
Clause 63: The method of clause 62, wherein the step of manufacturing the active pharmaceutical agent further comprises spray drying the final solution using nitrogen.
Clause 64: The method of clause 62 or 63, further comprising spray drying the final solution at an inlet temperature of from 82 °C to 150 °C, and with an outlet temperature of from 50°C to 90 °C.
Clause 65: An injector for delivering a solution of an active pharmaceutical agent and a diluent to a subject, the injector comprising:
a housing;
a spring release;
an outer cap separably engaged with the spring release and rotatable with the spring release;
a diluent ejector comprising a stopper;
a mixing spring adjacent the diluent ejector providing a bias to the diluent ejector within the housing;
a mixing-chamber seal;
a fluid chamber proximal to the stopper;
a powder chamber; and
a first tearable seal positioned between the fluid chamber and the powder chamber.
Clause 66: The injector of clause 65, wherein the diluent ejector is configured to translate proximally and at least partially tear the first tearable seal, thereby causing a diluent housed in the fluid chamber to enter the powder chamber and mix with an active pharmaceutical agent positioned within the powder chamber.
Clause 67: The injector of clause 65, wherein the first tearable seal is disposed at least partially within a stopper.
Clause 68: The injector of clause 65 further comprising a second tearable seal positioned between the powder chamber and the mixing-chamber seal.
Clause 69: The injector of clause 68 further comprising a fluid channel disposed between the second tearable seal and the mixing-chamber seal.
Clause 70: The injector of clause 65 further comprising:
a latch connected to the mixing-chamber seal; and
a needle hub engageable with the latch.
Clause 71: The injector of Clause 65 further comprising:
a needle;
a needle cap; and
a needle cap spring.
Clause 72: The injector of Clause 65, wherein the outer cap is twistable with the spring release from between 0° and 20°.
Clause 73: The injector of Clause 65, wherein the outer cap is twistable with the spring release from between 0° and 45°.
Clause 74: The injector of Clause 65, wherein the outer cap is twistable with the spring release from between 0° and 90°.
Clause 75: The injector of Clause 65, wherein the outer cap is twistable with the spring release from between 0° and 135°.
Clause 76: The injector of Clause 65, wherein the outer cap is twistable with the spring release from between 0° and 180°.
Clause 77: The injector of clause 65, wherein the outer cap is twistable with the spring release from between 0° and 225°.
Clause 78: The injector of Clause 65, wherein the outer cap is twistable with the spring release from between 0° and 270°.
Clause 79: The injector of Clause 65, wherein the outer cap is twistable with the spring release from between 0° and 315°.
Clause 80: The injector of Clause 65, wherein the outer cap is twistable with the spring release from between 0° and 360°.
Clause 81: The injector of any of Clauses 65 to 80, wherein twisting the outer cap comprises twisting the outer cap greater than 360°.
Clause 82: The injector of any of Clauses 65 to 81 further comprising an active pharmaceutical agent, wherein the active pharmaceutical agent is a spray-dried powder stored in the powder chamber.
Clause 83: The injector of clause 82, wherein the active pharmaceutical agent is a hydrocortisone sodium succinate sodium salt.
Clause 84: The injector of any of Clauses 65 to 83, wherein the outer cap is engageable with the housing to cover and protect a needle cap.
Clause 85: The injector of any of Clauses 65 to 84, wherein a cross section of the housing comprises a tear drop shape.
Clause 86: The injector of any of Clauses 65 to 85, wherein the housing comprises a powder-inspection window placed proximate a location of the powder chamber:
Clause 87: The injector of any of Clauses 65 to 86, wherein the housing comprises a powder-inspection window placed proximate a location of the powder chamber, the powder-inspection window providing a view of the powder chamber such that a first mixing of the active pharmaceutical agent and the diluent.
Clause 88: The injector of any of Clauses 65 to 87, wherein the housing comprises a reconstitution window placed within a wall of the housing and proximal to the powder chamber, the reconstitution window providing a view within the injector proximate a location of a final mixing of the active pharmaceutical agent and the diluent.
Clause 89: The injector of clause 88, wherein the housing further comprises a powder-inspection window placed proximate a location of the powder chamber, the powder-inspection window providing a view of the powder chamber such that a first mixing of the active pharmaceutical agent and the diluent.
Clause 90: The injector of any of Clauses 65 to 89 further comprising a spring container proximate the diluent ejector and engaged with the mixing spring, the spring container releasably engageable with the spring release, wherein disengagement of the spring container from the spring release releases the bias of the spring container to actuate the diluent ejector.
Clause 91: The injector of clause 90, wherein: the spring container comprises a circumferential groove; the spring release comprises a spring arm with a cam extending therefrom, the cam extending into the circumferential groove; and the spring arm is configured to deflect radially to cause the cam to disengage from the circumferential groove.
Clause 92: The injector of clause 91 further comprising a retaining clip engaged with the housing distal to the mixing spring, the retaining clip comprising a lever extension extending proximally and into the housing.
Clause 93: The injector of clause 92, wherein the lever extension is positioned proximate the spring arm and is engageable with the spring arm, wherein the spring arm is configured to engage with the lever extension in response to rotation of the spring release thereby causing radial deflection of the spring arm.

It is to be understood that the terminology used herein is for the purpose of describing particular embodiments only, and is not intended to limit the scope of the present disclosure which will be limited only by the appended claims.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about” or “approximately,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” or “approximately” as used herein when referring to a measurable value such as an amount, a temporal duration, and the like, is meant to encompass variations of +20%, ±10%, ±5%, ±1%, ±0.5%, or ±0.1% from the specified value, as such variations are appropriate to perform the disclosed methods.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

The term “pharmaceutically acceptable carrier” or “pharmaceutically acceptable diluent” as used herein is meant to refer to an excipient, carrier or diluent that can be administered to a subject, together with an agent or the pharmaceutical compositions disclosed herein, and which is inert or fails to eliminate the pharmacological activity of the active agent of the pharmaceutical composition. In some embodiments, the pharmaceutically acceptable carrier does fails to destroy or is incapable of eliminating the pharmacological activity of an active agent/vaccine and is nontoxic when administered in doses sufficient to deliver a therapeutic amount of the active agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH2)n—COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, PA, p. 1418(1985 ) . In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “subject,” “individual,” “host,” and “patient,” are used interchangeably herein and refer to a vertebrate individual, including but not limited to a mammal or human, for whom diagnosis, treatment, therapy, or drug injection is desired, particularly humans. Mammals include, but are not limited to, murines, simians, humans, farm animals, cows, pigs, goats, sheep, horses, dogs, sport animals, and pets. The methods described herein are applicable to both human therapy and veterinary applications. In some instances in the description of the present disclosure, the term “patient” refers to human patients suffering from a particular disease or disorder. In some embodiments, the subject is a mammal, and, in other embodiments, the subject is a human.

For any therapeutic agent described herein the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. The term “about” or “approximately” as used herein when referring to a measurable value such as an amount, a temporal duration, and the like, is meant to encompass variations of ±10%, ±5%, ±1%, or ±0.1% from the specified value, as such variations are appropriate to perform the disclosed methods. Where a numerical limitation is used, unless indicated otherwise by the context, “about” means the numerical value can vary by ±10%, ±5%, +4%, +3%, ±2%, or ±1% and remain within the scope of the disclosed embodiments.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included. As used herein, the terms “optional” or “optionally” mean that the subsequently described event or circumstance can or cannot occur, and that the description includes instances where said event or circumstance occurs and instances where it does not.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soy bean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

It should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

1. An injector for delivering a solution of an active pharmaceutical agent and a diluent to a subject, the injector comprising:
a housing;
a spring release;
an outer cap separably engaged with the spring release and rotatable with the spring release;
a diluent ejector comprising a stopper;
a mixing spring adjacent the diluent ejector providing a bias to the diluent ejector within the housing;
a powder barrel comprising a first tearable seal and a second tearable seal;
a crown of thorns positioned proximate the second tearable seal and configured to pierce the second tearable seal responsive to translation of the powder barrel proximally within the housing; and
a fluid chamber disposed between the stopper and the first tearable seal.

2. The injector of claim 1 further comprising a mixing-chamber seal movable within the housing responsive to a positive pressure caused by proximal translation of the diluent ejector via the mixing spring.

3. The injector of claim 2 further comprising:
a latch connected to the mixing-chamber seal; and
a needle hub engageable with the latch.

4. The injector of claim 2 further comprising:
a needle;
a needle cap; and
a needle cap spring.

5. The injector of claim 2, wherein the outer cap is twistable with the spring release from between 0° and 20°.

6. The injector of claim 2, wherein the outer cap is twistable with the spring release from between 0° and 45°.

7-14. (canceled)

15. The injector of claim 2 further comprising the active pharmaceutical agent disposed within the powder barrel.

16. The injector of claim 15, wherein the active pharmaceutical agent is a spray-dried powder stored in the powder barrel.

17. The injector of claim 16, wherein the active pharmaceutical agent is a hydrocortisone sodium succinate sodium salt.

18. The injector of claim 16, wherein the powder barrel comprises greater than 75 mg of the spray-dried powder.

19. The injector of claim 1, wherein the outer cap is engageable with the housing to cover and protect a needle cap.

20. The injector of claim 1, wherein a cross section of the housing comprises a tear drop shape.

21. The injector of claim 1, wherein the housing comprises a powder-inspection window placed proximate a location of the powder barrel.

22. The injector of claim 1, wherein the housing comprises a powder-inspection window placed proximate a location of the powder barrel, the powder-inspection window providing a view of the powder barrel before or during a first mixing of the active pharmaceutical agent and the diluent.

23. The injector of claim 1, wherein the housing comprises a reconstitution window placed within a wall of the housing and proximal to the crown of thorns, the reconstitution window providing a view within the injector proximate a location of a final mixing of the active pharmaceutical agent and the diluent.

24. The injector of claim 23, wherein the housing further comprises a powder-inspection window placed proximate a location of the powder barrel, the powder-inspection window providing a view of the powder barrel before or during a first mixing of the active pharmaceutical agent and the diluent.

25. The injector of claim 1 further comprising a spring container proximate the diluent ejector and engaged with the mixing spring, the spring container releasably engageable with the spring release, wherein disengagement of the spring container from the spring release releases the bias of the spring container to actuate the diluent ejector.

26. The injector of claim 25, wherein:
the spring container comprises a circumferential groove;
the spring release comprises a spring arm with a cam extending therefrom, the cam extending into the circumferential groove; and
the spring arm is configured to deflect radially to cause the cam to disengage from the circumferential groove.

27. The injector of claim 26 further comprising a retaining clip engaged with the housing distal to the mixing spring, the retaining clip comprising a lever extension extending proximally and into the housing.

28. The injector of claim 27, wherein the lever extension is positioned proximate the spring arm and is engageable with the spring arm, wherein the spring arm is configured to engage with the lever extension in response to rotation of the spring release thereby causing radial deflection of the spring arm.

29-93. (canceled)
